(19) United States
(12) Patent Application Publication
(10) Pub. No.: US 2013/0178845 A1
(43) Pub. Date: Jul. 11, 2013

(54) DEVICES AND METHODS FOR BIPOLAR AND MONOPOLAR PROCEDURES

(71) Applicants: Paul SMITH, Smithfield, RI (US); Samuel RAYBIN, Framingham, MA (US); Joseph LEVENDUSKY, Groton, MA (US); Naroun SUON, Lawrence, MA (US); Paul AQUILINO, South Walpole, MA (US)

(72) Inventors: Paul SMITH, Smithfield, RI (US); Samuel RAYBIN, Framingham, MA (US); Joseph LEVENDUSKY, Groton, MA (US); Naroun SUON, Lawrence, MA (US); Paul AQUILINO, South Walpole, MA (US)

(73) Assignee: BOSTON SCIENTIFIC SCIMED, INC., Maple Grove, MN (US)

(21) Appl. No.: 13/734,376

(22) Filed: Jan. 4, 2013

Related U.S. Application Data
(60) Provisional application No. 61/583,352, filed on Jan. 5, 2012.

(51) Int. Cl. A61B 18/18

(52) U.S. Cl. CPC A61B 18/18
USPC 606/33; 606/41; 606/46; 606/50

(57) ABSTRACT

An integrated catheter assembly comprises a bipolar electrode tool and a monopolar electrode tool. The catheter assembly enables an operator to perform both bipolar and monopolar procedures on tissue without having to withdraw the catheter assembly, without having to remove or replace any part of the catheter assembly, and/or without having to insert any additional tools or parts. The catheter assembly may comprise a switching mechanism such that when one of the bipolar electrode tool and monopolar electrode tool is electrically activated, the other of the bipolar electrode tool and monopolar electrode tool cannot be electrically activated. In one embodiment of a method, the operator uses a single catheter assembly for applying bipolar current for tissue electro-therapy and monopolar current for tissue cutting.

### CROSS REFERENCE TO RELATED APPLICATION

The present application claims priority to U.S. provisional application Ser. No. 61/583,352 filed Jan. 5, 2012, the disclosure of which is incorporated herein by reference in its entirety.

### FIELD OF THE INVENTION

This invention generally relates to devices and methods for bipolar and monopolar procedures.

### BACKGROUND OF THE INVENTION

As is known by those of skill in the art, radiofrequency (RF) energy of suitable current density and wave form may be used to seal potential hemorrhaging or bleeding areas by electro-coagulation of tissue and blood, without cutting. With this technique, RF coagulation current applied to the tissue generates heat by resistive losses in the conductive tissue. The resulting heat drives out extracellular and intracellular water resulting in coagulation necrosis. Similarly, the technique can be used to cause necrosis of (i.e., “ablate”) tissue, for example tissue that is performing improperly such as arrhythmic heart tissue.

One method of performing electro-coagulation and/or electro-cauterization of tissue is through the use of monopolar electrodes, in which one electrode is carried by a catheter to the site while the other electrode is an exterior ground plate placed on the skin of the patient. In another method, a bipolar catheter is employed. An example is the GOLD PROBE™ electrohemostasis catheter, manufactured by Boston Scientific Corporation, the assignee of the present invention. It comprises a flexible catheter with a distal tip formed of a ceramic cylinder having a hemispherical end. The ceramic tip includes a pair of gold spiral electrodes applied to its cylindrical surface and domed end. The spiral electrodes are separated by insulated areas in an arrangement resembling the stripes of a barber pole. RF current that flows from one electrode through the tissue contacted by the tip of the catheter to the other electrode heats and cauterizes the tissue. The catheter is constructed to be employed through the working channel of an endoscope to seal potential bleeding sites such as in the gastrointestinal (GI) tract or the esophagus.

RF electro-therapy catheters of other forms have been introduced through the vascular system to the heart to remedy arrhythmia. In this case, electrophysiological evaluation is performed at locations on the heart, and when a site requiring treatment is found, the catheter is used to ablate or deaden the tissue to correct the arrhythmia.

Other forms of treatment to address bleeding sites or sites requiring ablation have included the use of catheter-placed needles that inject drug agents such as vaso-constrictors for reducing bleeding and absolute ethanol for ablation of tissue.

Physicians often use different catheters to perform different functions. For example, physicians often use one catheter to perform irrigation and hemostasis and another to make an injection or to cut tissue. The exchange of catheters to provide different functions extends the time to complete the therapy, increases the risk to the patient and also increases patient discomfort.

U.S. Pat. No. 5,403,311, the contents of which are incorporated herein by reference, discloses an example of a catheter device for bipolar electro-coagulation. U.S. Pat. No. 5,336,222, the contents of which are incorporated herein by reference, discloses an integrated catheter assembly for enabling diverse in situ therapies which includes an irrigation fluid lumen, a distal tip portion that acts as a hemostat and a needle for injection therapy. U.S. Pat. No. 6,325,800, the contents of which are incorporated herein by reference, discloses a catheter assembly which includes an irrigation fluid lumen, a distal tip portion that acts as a hemostat and an electrical cutting wire.

### SUMMARY OF THE INVENTION

In accordance with this invention, an integrated catheter assembly is provided that enables both bipolar and monopolar procedures. For example, the integrated catheter assembly may enable bipolar coagulation as well as monopolar cutting of tissue.

In one embodiment, a catheter assembly comprises a bipolar electrode tool and a monopolar electrode tool. In one embodiment, the bipolar electrode tool and the monopolar electrode tool are part of a single integrated instrument. The catheter assembly enables an operator to perform both bipolar and monopolar procedures without having to withdraw the catheter assembly from the working channel or lumen of an endoscope, without having to remove or replace any part of the catheter assembly and without having to insert any additional tools or parts.

The catheter assembly may further comprise a switching mechanism such that when one of the bipolar electrode tool and the monopolar electrode tool is electrically activated, the other of the bipolar electrode tool and the monopolar electrode tool cannot be electrically activated.

The catheter assembly may comprise a handle, and the switching mechanism may be located in the handle. The switching mechanism may comprise fixed contacts that are engaged or disengaged by movement of one or more moveable contacts on the monopolar electrode tool or the bipolar electrode tool.

The catheter assembly may further comprise a position engagement element moveable with the monopolar electrode tool or the bipolar electrode tool, wherein the position engagement element is adapted to engage a corresponding position engagement structure to selectively lock the relative positions of the monopolar electrode tool and the bipolar electrode tool. The position engagement element and position engagement structure may be adapted to allow the monopolar electrode tool to be moved between and locked into at least an extended position and a retracted position.

In certain embodiments, retraction of the monopolar electrode tool renders the bipolar circuit active and the monopolar circuit inactive, and extension of the monopolar electrode tool renders the bipolar circuit inactive and the monopolar circuit active. That is, when the monopolar electrode tool is in the extended position, the monopolar electrode tool may be rendered capable of being electrically activated, and the bipolar electrode tool may be rendered incapable of being electrically activated. When the monopolar electrode tool is in the retracted position, the bipolar electrode tool may be rendered capable of being electrically activated, and the monopolar electrode tool may be rendered incapable of being electrically activated.

In one embodiment of a method, the operator (e.g., physician) may use a single catheter assembly for applying bipolar current for one tissue procedure and monopolar current for another tissue procedure. The method may comprise inserting a catheter assembly into a working channel or an endoscope at a desired location in a patient's body. The catheter assembly may comprise a bipolar electrode tool and a monopolar electrode tool. The catheter assembly may also comprise a switching mechanism adapted such that when one of the bipolar electrode tool and monopolar electrode tool is electrically activated, the other of the bipolar electrode tool and monopolar electrode tool cannot be electrically activated.

### BRIEF DESCRIPTION OF THE DRAWINGS

The various objects, advantages and novel features of this invention will be more fully apparent from a reading of the following detailed description in conjunction with the accompanying drawings in which like reference numerals refer to like parts, and in which:

FIG. 1 is a side view, partially in section, of an integrated catheter assembly according to a first embodiment.

FIG. 2 is a perspective view of a portion of the cutting tool and switching mechanism of the integrated catheter assembly of FIG. 1.

FIG. 3 is a side view, partially in section, of an integrated catheter assembly according to a second embodiment.

### DETAILED DESCRIPTION

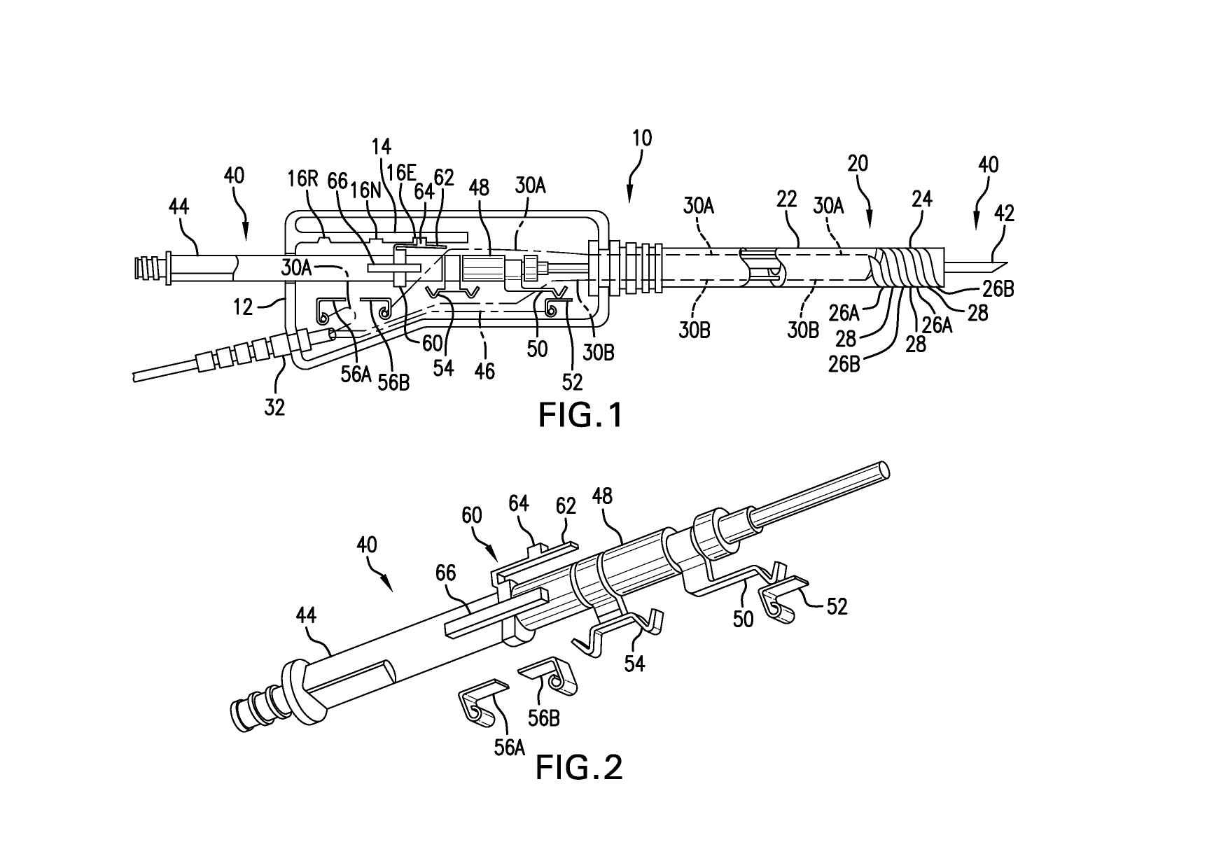

FIG. 1 shows an intervention apparatus according to a first embodiment, comprising a catheter assembly 10 having a bipolar electrode tool 20 and a monopolar electrode tool 40. In this illustrated embodiment the monopolar electrode tool 40 is a cutting tool, but the monopolar electrode tool 40 may be adapted for other electro-procedures. The catheter assembly 10 of FIG. 1 enables a physician to perform both bipolar electro-procedures (e.g., coagulation/cauterization) as well as monopolar electro-procedures (e.g., cutting of tissue) without having to withdraw the catheter assembly 10 from the working channel or lumen of an endoscope, without having to remove or replace any part of the device and without having to insert any additional tools or parts.

The bipolar electrode structure of the bipolar electrode tool 20 may be similar at least in some respects to the bipolar electrode structure of the above-identified GOLD PROBE™ electrohemostasis catheter and of U.S. Pat. No. 5,403,311. It comprises a flexible catheter 22 with a distal tip 24 formed of a ceramic cylinder. In the embodiment of FIG. 1, the distal tip 24 has a flat distal end, but it will be appreciated that the distal end may have other shapes such as rounded or hemispherical or any other appropriate shape. The distal tip 24 includes a pair of spiral electrodes 26A and 26B applied to its cylindrical surface. The spiral electrodes may be formed of any suitable conductive material, such as gold or another suitable metal. The spiral electrodes 26A and 26B are separated by insulated areas 28 in an arrangement resembling the stripes of a barber pole. It will be appreciated that the distal tip of the bipolar electrode tool may have more than two electrodes spaced with any suitable geometry. The electrodes are not limited to spiral electrodes. Alternative embodiments include, but are not limited to, rings, tabs (e.g., square tabs), lines, etc.

Bipolar electrical lead wires 30A and 30B extend through the catheter assembly 10 and are connected at their distal ends to the spiral electrodes 26A and 26B, respectively. At the proximal end of the device, the lead wires 30A and 30B extend through a lead wire hub 32 to an RF generator connector (not shown). The RF generator connector may be connected to an RF generator, as is well-known in the art, for supplying bipolar current through the lead wires 30A and 30B and the spiral electrodes 26A and 26B. When the distal tip 24 of the catheter assembly 10 is placed against tissue with the cutting tool 40 in a retracted position (as described below), current applied through the lead wires 30A and 30B flows between the electrodes 26A and 26B through the contacted tissue and heats and cauterizes the tissue.

The catheter assembly 10 includes a handle or housing 12. The lead wire hub 32 is connected to the handle 12 at a proximal end of the handle 12, and the flexible catheter 22 is connected to the handle 12 at a distal end of the handle 12. The lead wires 30A and 30B extend through the handle 12 from the lead wire hub 32 to the flexible catheter 22. Inside the handle 12, the lead wires 30A and 30B are connected through a switching mechanism, described in more detail below.

The monopolar cutting tool 40 includes an electrical cutting element 42 made of an electrically conductive material. The cutting element 42 may be a needle in order to allow injections of fluids or drugs and/or to apply suction. The cutting element 42 extends through a lumen inside the flexible catheter 22 of the bipolar electrode tool 20 and inside the distal tip 24 of the bipolar electrode tool 20. The cutting element 42 can be moved by an operator between extended and retracted positions by manipulation of a cutting element actuator 44. In FIG. 1, the cutting tool 40 is shown with the cutting element 42 in the extended position, in which the distal-most portion of the cutting element 42 projects through an opening in the distal end of the distal tip 24 of the bipolar electrode tool 20. A monopolar electrical lead wire 46 extends through the lead wire hub 32 to supply monopolar electrical current to the electrical cutting element 42. The lead wire 46 extends through the lead wire hub 32 to the RF generator connector (not shown). The RF generator connector may be connected to an RF generator as mentioned above, which can supply monopolar current through the lead wire 46 (and also can supply bipolar current through the lead wires 30A and 30B, as described above).

The cutting element 42 may be conductive along its entire length or only a distal portion of the cutting element 42 may be conductive, in which case a conductive element is arranged to connect the lead wire 46 to the distal portion of the cutting element 42. Additionally, if desired, substantially all but the distal end of the cutting element 42 may be coated or covered, e.g., with an insulating material. The cutting element 42 and its current pathway is electrically isolated from the bipolar electrodes 26A and 26B and their current pathway. The cutting element 42 may be solid or may be a hollow needle as mentioned above, in which case the lumen of the cutting element 42 can be used to allow passage of fluids for injection. The actuator 44 can have a Luer-type fitting which allows passage of fluids for injection.

As can be seen in FIGS. 1 and 2, the cutting tool 40 has electrical contacts 50, 54 mounted on it to allow switching between supplying current through the spiral electrodes 26A and 26B and supplying current to the cutting element 42. Attached to the cutting tool 40 and directly connected to the cutting element 42 is a shiftable cutting element contact 50. In this illustrated embodiment, the shiftable cutting element contact 50 is in direct electrical connection with the cutting element 42. Attached to the cutting tool 40 and electrically insulated from the cutting element 42 is a shiftable bipolar electrode contact 54. The cutting tool 40 may have insulating material 48 around a portion of the length of the cutting element 42. The shiftable bipolar electrode contact 54 may be mounted over the insulating material 48 in order to electrically insulate the shiftable bipolar electrode contact 54 from the cutting element 42.

Mounted inside the housing 12 is a fixed cutting element contact 52 and fixed bipolar electrode contacts 56A and 56B. The fixed cutting element contact 52 is connected to monopolar electrical lead wire 46. The fixed bipolar electrode contacts 56A and 56B are connected to the bipolar electrical lead wire 30A. That is, the electrical lead wire 30A has a gap in it, with contact 56A connected to the electrical lead wire 30A on one side of the gap and contact 56B connected to the electrical lead wire 30A on the other side of the gap.

In addition to the shiftable cutting element contact 50 and the shiftable bipolar electrode contact 54, also mounted on the cutting tool 40 is a position engagement element 60 which comprises a flexible spring arm 62 and a tab 64. The housing 12 includes a corresponding position engagement structure 14 that comprises notches 16E, 16N and 16R, as shown. The position engagement element 60 of the cutting tool 40 interacts with the position engagement structure 14 of the housing 12 in order hold the cutting tool 40 in one of three positions, as selected by the operator. The spring arm 62, tab 64, and notches 16E, 16N and 16R are designed such that they can hold the cutting tool 40 in position until overcome by sufficient force applied to the cutting element actuator 44 to move the cutting tool 40 to another position. That is, the natural bias of the flexible spring arm 62 presses the tab 64 into one of the notches 16E, 16N or 16R. A sufficient axial force applied to the cutting tool 40 via the cutting element actuator 44 will cause the flexible spring arm 62 to flex inwardly (toward the shaft of the cutting tool 40), thereby permitting the tab 64 to disengage from its corresponding notch in order to allow the cutting tool to be moved proximally and/or distally to another position.

When the cutting tool 40 is moved by the operator to its distal-most or extended position, which is the position shown in FIG. 1, the tab 64 engages the notch 16E. In this position, the cutting element 42 is extended with the distal-most portion of the cutting element 42 projecting through the opening in the distal end of the distal tip 24 of the bipolar electrode tool 20. In this position, the shiftable cutting element contact 50 engages the fixed cutting element contact 52. With this engagement, the monopolar current path is closed, and monopolar current is allowed to flow through the monopolar electrical lead wire 46 to the cutting element 42. In this position, the shiftable bipolar electrode contact 54 does not bridge the gap between fixed bipolar electrode contacts 56A and 56B, and, therefore, the bipolar current path is open, and electrical current is prevented from flowing through the spiral electrodes 26A and 26B.

When the cutting tool 40 is moved by the operator to its middle or neutral position, the tab 64 engages the notch 16N. In this position, the distal-most portion of the cutting element 42 is pulled to be within the lumen of the flexible catheter 22 so that it is not exposed. In this position, the shiftable cutting element contact 50 no longer engages the fixed cutting element contact 52. Therefore, the monopolar current path is open, and electrical current is prevented from flowing to the cutting element 42. In addition, in this position, the shiftable bipolar electrode contact 54 does not bridge the gap between fixed bipolar electrode contacts 56A and 56B, and, therefore, the bipolar current path is open, and electrical current is also prevented from flowing through the spiral electrodes 26A and 26B.

When the cutting tool 40 is moved by the operator to its proximal-most or retracted position, the tab 64 engages the notch 16R. In this position, the distal-most portion of the cutting element 42 is pulled to be further within the lumen of the flexible catheter 22, again not exposed. In this position, the shiftable bipolar electrode contact 54 now contacts both of the fixed bipolar electrode contacts 56A and 56B. In this manner, the shiftable bipolar electrode contact 54 bridges the gap between fixed bipolar electrode contacts 56A and 56B. With this engagement, the bipolar current path is closed, and, therefore, electrical current is allowed to flow through the electrical lead wire 30A, through the spiral electrodes 26A and 26B (and tissue), and through the electrical lead wire 30B. In this position, as in the neutral position, the shiftable cutting element contact 50 does not engage the fixed cutting element contact 52, and, therefore, the monopolar current path is open, and electrical current is prevented from flowing to the cutting element 42.

As can be appreciated, this switching mechanism allows the electrical activation of the bipolar electro-therapy tip and the monopolar cutting element to be controlled by the operator's extension and retraction of the cutting element. When the cutting element is fully extended, the monopolar current path is closed, current can be supplied to the cutting element in order for it to be operated as a monopolar cutting tool, and current to the bipolar electro-therapy tip is cut off. When the cutting element is fully retracted, the bipolar current path is closed, current can flow through the bipolar electro-therapy tip, and current to the monopolar cutting element is cut off.

It will be appreciated that the catheter assembly can include appropriate seals (not shown) in order to prevent fluid from entering undesired areas, for example areas where fluid could close a current path or otherwise interfere with the desired functioning. In alternate embodiments (not shown), instead of internal switching components controlled by tool movement, current may be controlled by one or more switches in or on the housing that allow either monopolar or bipolar activation and prevent the other. For example, a switch on the housing may be manually operable to switch the current. In addition, the catheter assembly may further include a reset spring or the like to assist in resetting the device to an original position or a neutral position.

As shown in FIGS. 1 and 2, the cutting tool 40 carries a guide element 66 that may mate with a corresponding groove (not shown) in the housing. In this manner, the movement of the cutting tool 40 can be stabilized, allowing movement of the cutting tool 40 in the axial direction but preventing rotational movement of the cutting tool 40 within the housing. A similar result can be achieved using a groove on the cutting tool and a corresponding projecting guide element integral with the housing. Stop elements can be provided to prevent the cutting tool from being moved proximally beyond its proximal-most position or distally beyond its distal-most position.

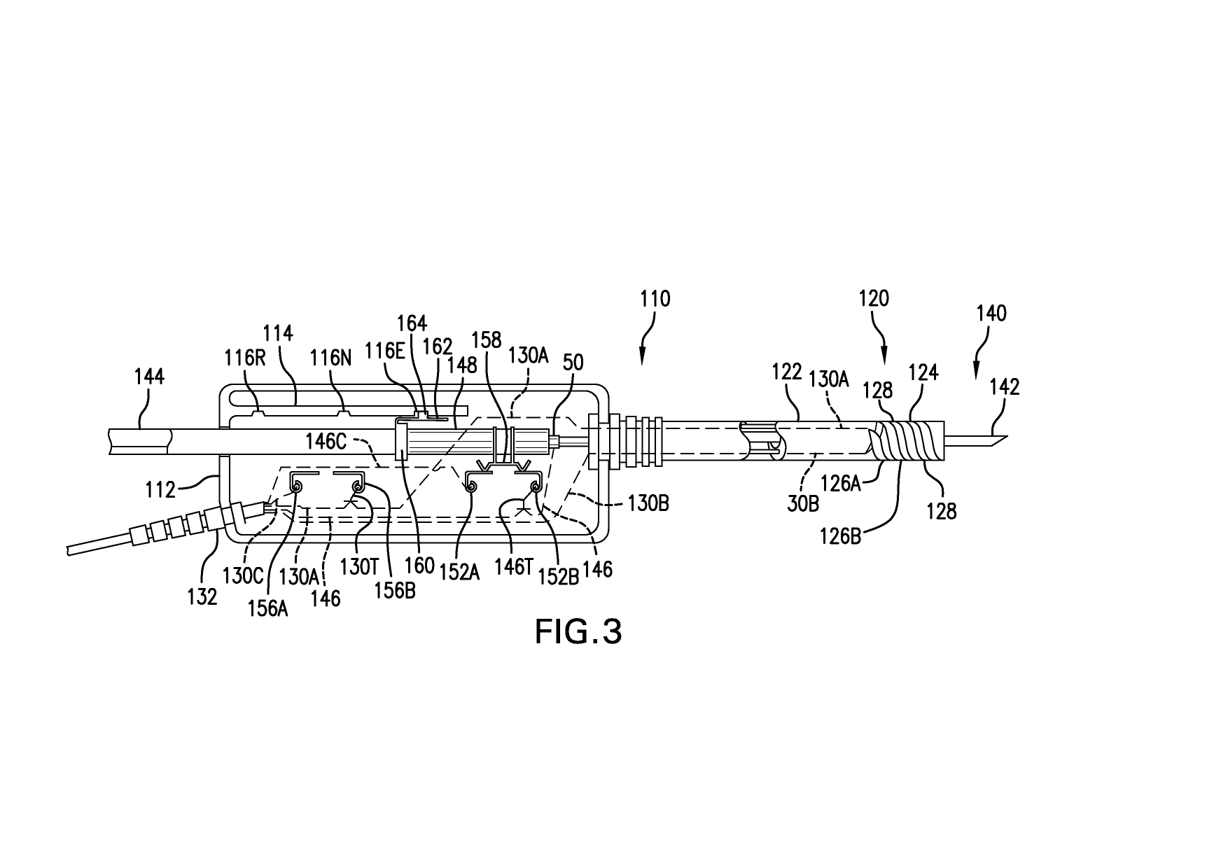

FIG. 3 shows an intervention apparatus according to a second embodiment comprising a catheter assembly 110 having a bipolar electrode tool 120 and a monopolar cutting tool 140. The catheter assembly 110 of FIG. 3, like the catheter assembly 10 of FIG. 1, enables a physician to perform both bipolar electro-therapy (coagulation/cauterization) and monopolar cutting of tissue without having to withdraw the catheter assembly 110 from the working channel or lumen of an endoscope, without having to remove or replace any part of the device, and without having to insert any additional tools or parts.

The bipolar electrode tool 120 is similar to the bipolar electrode tool 20 of FIG. 1, comprising a flexible catheter 122 and a distal tip 124. The distal tip 124 includes a pair of spiral electrodes 126A and 126B separated by insulated areas 128. These elements of bipolar electrode tool 120 may be the same as the corresponding elements of bipolar electrode tool 20.

Bipolar electrical lead wires 130A and 130B extend through the catheter assembly 110 and are connected at their distal ends to the spiral electrodes 126A and 126B, respectively. At the proximal end of the device, the lead wires 130A and 130B extend through a lead wire hub 132 to an RF generator connector (not shown). The RF generator connector may be connected to an RF generator, as is well-known in the art, for supplying bipolar current through the lead wires 130A and 130B and the spiral electrodes 126A and 126B. When the distal tip 124 of the catheter assembly 110 is placed against tissue with the cutting tool 140 in a refracted position (as described below), current applied through the lead wires 130A and 130B flows between the electrodes 126A and 126B through the contacted tissue and heats and cauterizes the tissue.

The catheter assembly 110 includes a handle or housing 112. The lead wire hub 132 is connected to the handle 112 at a proximal end of the handle 112, and the flexible catheter 122 is connected to the handle 112 at a distal end of the handle 112. The lead wires 130A and 130B extend through the handle 112 from the lead wire hub 132 to the flexible catheter 122. Inside the handle 112, the lead wires 130A and 130B are connected through a switching mechanism, as described in more detail below.

The cutting tool 140 includes an electrical cutting element 142 made of an electrically conductive material similar to the cutting element 142 described above. The cutting element 142 may be a needle in order to allow injections of fluids or drugs and/or to apply suction. The cutting element 142 extends through a lumen inside the flexible catheter 122 of the bipolar electrode tool 120 and inside the distal tip 124 of the bipolar electrode tool 120. The cutting element 142 can be moved by an operator between extended and refracted positions by manipulation of a cutting element actuator 144. In FIG. 3, the cutting tool 140 is shown with the cutting element 142 in the extended position, in which the distal-most portion of the cutting element 142 projects through an opening in the distal end of the distal tip 124 of the bipolar electrode tool 120. A monopolar electrical lead wire 146 extends through the lead wire hub 132 to supply monopolar electrical current to the electrical cutting element 142. In FIG. 3, the distal end of electrical lead wire 146 is directly connected to a ring 150 mounted on the cutting element 142, so that the electrical lead wire 146 is electrically coupled to the cutting element 142. The proximal end of the lead wire 146 extends through the lead wire hub 132 to the RF generator connector (not shown). The RF generator connector may be connected to an RF generator as mentioned above, which can supply monopolar current through the lead wire 146 (and also can supply bipolar current through the lead wires 130A and 130B, as described above).

The cutting element 142 may be conductive along its entire length or only a distal portion of the cutting element 142 may be conductive, in which case a conductive element is arranged to connect the lead wire 146 to the distal portion of the cutting element 142. Additionally, if desired, substantially all but the distal end of the cutting element 142 may be coated or covered, e.g., with an insulating material. The cutting element 142 and its current pathway is electrically isolated from the bipolar electrodes 126A and 126B and their current pathway. The cutting element 142 may be solid or may be a hollow needle as mentioned above, in which case the lumen of the cutting element 142 can be used to allow passage of fluids for injection. The actuator 144 can have a Luer-type fitting which allows passage of fluids for injection.

As can be seen in FIG. 3, current flow through the electrical lead wire 130A and through the electrical lead wire 146 is controlled by a switching mechanism. First, with reference to the monopolar element, a transistor 146T regulates current through the lead wire 146. A lead wire 146C is connected to a first monopolar contact 152A which is separated by a gap from second monopolar contact 152B. The second monopolar contact 152B is in turn connected to the transistor 146T. Similarly, with reference to the bipolar element, a transistor 130T regulates current through the lead wire 130A. A lead wire 130C is connected to a first bipolar contact 156A which is separated by a gap from second bipolar contact 156B. The second bipolar contact 156B is in turn connected to the transistor 130T. The contacts 152A, 152B, 156A and 156B are fixed relative to the housing 112.

The cutting tool 140 has bridging contact 158 mounted on it to allow switching between supplying current through the spiral electrodes 126A and 126B and supplying current to the cutting element 142. The cutting tool 140 may have insulating material 148 around a portion of the length of the cutting element 142. The bridging contact 158 may be mounted over the insulating material 148 in order to electrically insulate the bridging contact 158 from the cutting element 142.

Also mounted on the cutting tool 140 is a position engagement element 160 which comprises a flexible spring arm 162 and a tab 164. The housing 112 includes a corresponding position engagement structure 114 that comprises notches 116E, 116N and 116R, as shown. The position engagement element 160 of the cutting tool 140 interacts with the position engagement structure 114 of the housing 112 in order hold the cutting tool 140 in one of three positions, as selected by the operator. The spring arm 162, tab 164, and notches 116E, 116N and 116R are designed such that they can hold the cutting tool 140 in position until overcome by sufficient force applied to the cutting element actuator 144 to move the cutting tool 140 to another position, similar to the operation of the spring arm 62, tab 64 and notches 16E, 16N and 16R described above.

When the cutting tool 140 is moved by the operator to its distal-most or extended position, which is the position shown in FIG. 3, the tab 164 engages the notch 116E. In this position, the cutting element 142 is extended with the distal-most portion of the cutting element 142 projecting through the opening in the distal end of the distal tip 124 of the bipolar electrode tool 120. In this position, the bridging contact 158 contacts both of the fixed monopolar electrode contacts 152A and 152B. In this manner, the bridging contact 158 bridges the gap between fixed monopolar electrode contacts 152A and 152B, and, therefore, a voltage is allowed to be applied through lead wire 146C to the transistor 146T. When such a voltage is applied, current is then allowed to flow through lead wire 146 to the monopolar cutting element 142. In this position, the gap between fixed bipolar electrode contacts 156A and 156B remains unbridged, and, therefore, electrical current is prevented from flowing through the spiral electrodes 126A and 126B.

When the cutting tool 140 is moved by the operator to its middle or neutral position, the tab 164 engages the notch 116N. In this position, the distal-most portion of the cutting element 142 is pulled to be within the lumen of the flexible catheter 122 so that it is not exposed. In this position, the bridging contact 158 no longer electrically connects the fixed monopolar electrode contacts 152A and 152B. Therefore, electrical current is prevented from flowing to the cutting element 142. In addition, in this position, the bridging contact 158 does not bridge the gap between fixed bipolar electrode contacts 156A and 156B, and, therefore, electrical current is also prevented from flowing through the spiral electrodes 126A and 126B.

When the cutting tool 140 is moved by the operator to its proximal-most or retracted position, the tab 164 engages the notch 116R. In this position, the distal-most portion of the cutting element 142 is pulled to be further within the lumen of the flexible catheter 122, again not exposed. In this position, the bridging contact 158 now contacts both of the fixed bipolar electrode contacts 156A and 156B, bridging the gap between them. Accordingly, a voltage is allowed to be applied through lead wire 130C to the transistor 130T. When such a voltage is applied, current is then allowed to flow through lead wire 130A and through the spiral electrodes 126A and 126B and the electrical lead wire 130B. In this position, as in the neutral position, the bridging contact 158 does not bridge the gap between fixed monopolar electrode contacts 152A and 152B, and, therefore, electrical current is prevented from flowing to the cutting element 142.

As can be appreciated, similar to the switching mechanism of the catheter assembly 10, this switching mechanism of the catheter assembly 110 allows the electrical activation of the bipolar electro-therapy tip and the monopolar cutting element to be controlled by the operator's extension and retraction of the cutting element. When the cutting element is fully extended, current can be supplied to the cutting element in order for it to be operated as a monopolar cutting tool, and current to the bipolar electro-therapy tip is cut off. When the cutting element is fully retracted, current can flow through the bipolar electro-therapy tip, and current to the monopolar cutting element is cut off.

Similar to the catheter assembly 10, the catheter assembly 110 may have a guide element carried by the cutting tool that may mate with a corresponding groove in the housing to stabilize movement of the cutting tool. A similar result can be achieved using a groove on the cutting tool and a corresponding projecting guide element integral with the housing. Stop elements can be provided to prevent the cutting tool from being moved proximally beyond its proximal-most position or distally beyond its distal-most position.

Some examples and possible variations of methods of use of the catheter assemblies 10 and 110 are as follows.

In one example procedure, a physician will, as in the prior art, insert an endoscope with a working channel in the desired location, for example in the gastrointestinal tract or esophagus. The physician then can insert the catheter assembly 10, 110 through the working channel, by inserting the flexible catheter 22, 122 through the working channel. For such insertion, the cutting element 42, 142 may be retracted, for example with the assembly in the neutral position (notches 16N, 116N). This can prevent skiving of the working channel of the endoscope and also can allow for unobstructed operation of the bipolar electrode tip 24, 124.

If, upon viewing the site, the physician decides to utilize hemostasis, the physician can position the bipolar electrode tip 24, 124 at the tissue, move the cutting tool 40, 140 to the retracted position (notches 16R, 116R)—to move the switching mechanism into a position that allows current flow to the bipolar element—and then send bipolar current through the electrodes 26A, 126A and 26B, 126B. If the physician determines that cutting tissue and/or making an incision in the tissue is appropriate before, after or in lieu of hemostasis, the physician can extend the cutting element 42, 142 to the extended position (notches 16E, 116E) and then send monopolar current through the cutting element 42, 142 (with a suitably placed exterior electrode) to use the cutting element 42, 142 to cut or to make a surgical incision in the tissue. Each of these functions can be performed without withdrawing the integrated catheter assembly 10, 110 from the endoscope.

In one exemplary order of steps, the physician uses the bipolar element for coagulation and to mark a lesion. Then, the physician may extend the cutting element—which in this embodiment is in the form of a needle—and use it to inject fluid (e.g., saline) beneath the tissue in order to create a bleb and raise the lesion. Thereafter, the physician may apply current to the monopolar cutting element and use it to dissect the lesion.

In another exemplary order of steps, the physician extends the cutting element—which in this embodiment is in the form of a needle—and uses it for suction in order to remove blood from the site. Then, the physician retracts the needle and uses the bipolar element for coagulation. Thereafter, the physician may again extend the cutting element to inject fluid (e.g., saline) beneath the tissue and then apply monopolar current to it to cut the tissue.

In an example of specific embodiments and sizes, the outer diameter of the flexible catheter 22, 122 can be as small as 5 Fr. or less and as large as can be accommodated by the inner diameter of an endoscopic channel or guide. In certain specific embodiments, for example, the flexible catheter 22, 122 can comprise a 7 Fr. or 10 Fr. catheter.

Although this invention has been described in terms of specific embodiments and certain modifications, still other modifications can be made. For example, the catheter assembly may include an irrigation lumen and may be connected to an irrigation hub, as in U.S. Pat. No. 5,336,222. Conductive parts may be made of suitable conductive material and may be insulated as desired. The monopolar electrode tool is not limited to a probe or needle (solid or hollow) but may be, for example, a blade, hook, snare, loop, paddle, or other suitable structure. In some embodiments, the catheter assembly may comprise further lumens and/or further instruments, and such further instruments may or may not be adapted for electro-procedures. In an least one alternate embodiment, the catheter assembly comprises a monopolar cutting element and an additional snare. It will be apparent that these and other modifications can be made to the disclosed apparatus without departing from the invention.

What is claimed is:

1. A catheter assembly comprising:
a bipolar electrode tool comprising a distal tip with a plurality of electrodes located on the distal tip, wherein the bipolar electrode tool is adapted to apply bipolar current to tissue; and
a monopolar electrode tool comprising an electrode and being adapted to apply monopolar current to tissue;
wherein the catheter assembly further comprises a switching mechanism such that when one of the bipolar electrode tool and monopolar electrode tool is electrically activated, the other of the bipolar electrode tool and monopolar electrode tool cannot be electrically activated.

2. The catheter assembly of claim 1, wherein the catheter assembly further comprises a handle, and the switching mechanism is located in the handle.

3. The catheter assembly of claim 1, wherein the monopolar electrode tool and bipolar electrode tool have a first relative positioning and a second relative positioning, wherein the switching mechanism allows current to flow to the monopolar electrode tool in the first relative positioning and to the bipolar electrode tool in the second relative positioning.

4. The catheter assembly of claim 1, wherein the catheter assembly enables an operator to perform both a bipolar procedure and a monopolar procedure without having to withdraw the catheter assembly from the working channel or lumen of an endoscope.

5. The catheter assembly of claim 1, wherein the catheter assembly enables an operator to perform both a bipolar procedure and a monopolar procedure without having to remove or replace any part of the catheter assembly and without having to insert any additional tools or parts.

6. The catheter assembly of claim 1, further comprising bipolar electrical lead wires connected to the electrodes of the bipolar electrode tool and a monopolar lead wire connected to the electrode of the monopolar electrode tool.

7. The catheter assembly of claim 6, wherein the bipolar electrical lead wires and the monopolar lead wire are electrically coupled to one or more RF generator connectors.

8. The catheter assembly of claim 1, wherein the monopolar electrode tool comprises a cutting element.

9. The catheter assembly of claim 1, wherein when the monopolar electrode tool is in an extended position, the monopolar electrode tool is capable of being electrically activated, and the bipolar electrode tool is incapable of being electrically activated.

10. The catheter assembly of claim 1, wherein when the monopolar electrode tool is in a fully retracted position, the bipolar electrode tool is capable of being electrically activated, and the monopolar electrode tool is incapable of being electrically activated.

11. A catheter assembly comprising:
a bipolar electrode tool comprising a distal tip with a plurality of electrodes located on the distal tip, wherein the bipolar electrode tool is adapted to apply bipolar current to tissue; and
a monopolar electrode tool comprising an electrode and being adapted to apply monopolar current to cutting tissue;
wherein the catheter assembly further comprises a position engagement element, wherein the monopolar electrode tool and bipolar electrode tool have a first relative positioning and a second relative positioning, and wherein the position engagement element is adapted to engage a corresponding position engagement structure in order to selectively lock the relative positions of the monopolar electrode tool and the bipolar electrode tool in the first relative positioning and the second relative positioning.

12. The catheter assembly of claim 11, wherein the catheter assembly further comprises a handle, and the position engagement structure is located in the handle.

13. The catheter assembly of claim 11, wherein the position engagement element and position engagement structure are adapted to allow the monopolar electrode tool to be moved between and locked into at least a monopolar electrode tool extended position corresponding to the first relative positioning and a monopolar electrode tool retracted position corresponding to the second relative positioning.

14. The catheter assembly of claim 13, wherein when the monopolar electrode tool is in the extended position, the monopolar electrode tool is capable of being electrically activated, and the bipolar electrode tool is incapable of being electrically activated.

15. The catheter assembly of claim 13, wherein when the monopolar electrode tool is in the retracted position, the bipolar electrode tool is capable of being electrically activated, and the monopolar electrode tool is incapable of being electrically activated.

16. A method of applying bipolar current and monopolar current, the method comprising:
inserting an endoscope with a working channel in a desired location in a patient's body; and
inserting a catheter assembly through the working channel, the catheter assembly comprising:
a bipolar electrode tool comprising a distal tip with a plurality of electrodes located on the distal tip, wherein the bipolar electrode tool is adapted to apply bipolar current to tissue;
a monopolar electrode tool comprising an electrode and being adapted to apply monopolar current to tissue; and
a switching mechanism adapted such that when one of the bipolar electrode tool and monopolar electrode tool is electrically activated, the other of the bipolar electrode tool and monopolar electrode tool cannot be electrically activated.

17. The method of claim 16, further comprising using the bipolar electrode tool to perform coagulation while the monopolar electrode tool is in a retracted position.

18. The method of claim 16, further comprising extending the monopolar electrode tool and applying electrical current to the monopolar electrode tool in order to cut tissue.

19. The method of claim 16, further comprising the step of extending the monopolar electrode tool, wherein the step of extending the monopolar electrode tool closes an electrical connection from a proximal RF connector to the electrode of the monopolar electrode tool.

20. The method of claim 16, further comprising the step of retracting the monopolar electrode tool, wherein the step of retracting the monopolar electrode tool closes an electrical connection from a proximal RF connector to the electrodes of the bipolar electrode tool.
